(19) United States
(12) Patent Application Publication
(10) Pub. No.: US 2003/0027155 A1
(43) Pub. Date: Feb. 6, 2003

(54) Homozygous deletion of chromosome 8p23 in hepatocellular carcinoma

(76) Inventors: Anne DeJean, Paris (FR); Agnes Marchio, Sevran (FR); Pascal Pineau, Paris (FR)

Correspondence Address: FINNEGAN, HENDERSON, FARABOW,, GARRETT & DUNNER, L.L.P., 1300 I STREET, N.W., WASHINGTON, DC, 20005-3315, US

(21) Appl. No.: 09/950,840

(22) Filed: Sep. 13, 2001

Related U.S. Application Data
(60) Provisional application No. 60/234,308, filed on Sep. 21, 2000.

(51) Int. Cl. C12Q 1/68

(52) U.S. Cl. 435/6

(57) ABSTRACT

A method for detecting human hepatocellular carcinoma (HCC), wherein the method comprises detecting a homozygous deletion in human chromosome 8p23.

### CROSS-REFERENCE TO RELATED APPLICATIONS

This application is based on and claims the benefit of U.S. Provisional Application Ser. No. 60/234,308, filed Sep. 21, 2000 (Attorney Docket No. 03495.6050). The entire disclosure of this Provisional application is relied upon and incorporated by reference herein.

### BACKGROUND OF THE INVENTION

The identification of several major tumor suppressor genes (TSG) in the last decade has brought substantial gains in the fundamental understanding of two common human malignant tumors: breast and colorectal cancers. Notably, advances in molecular genetics lead to a better characterization of biological behaviors of tumors associated with specific TSG mutations (Kinzler and Vogelstein, 1997, Dillon et al., 1998). Ultimately, such progress would benefit patients at high risk of recurrence or metastasis and lead to the definition of appropriate treatments. However, such a propitious situation is not found in the field of primary liver cancer. Actually, major TSG involved in hepatocellular carcinoma (HCC) development and located on chromosomes 8p, 4q, 13q and 6q still remain to be identified.

Hepatocellular carcinoma (HCC), the most frequent histological form of primary liver cancer, is one of the most prevalent human tumors with more than 400,000 new cases diagnosed each year worldwide (Parkin et al., 1999). The natural history of HCC, generally diagnosed in an advanced form, is characterized by very poor survival rates (Markovic et al., 1998). Several studies have recently demonstrated a significant increase in its incidence in the past twenty years in Japan, USA, and Europe (Taylor-Robinson et al., 1997, Deuffic et al., 1998, El-Serag and Mason, 1999, Makimoto and Higuchi, 1999). This increased incidence is thought to reflect the strong impact of some infectious or environmental factors on the pathogenesis of the tumor. The importance of chronic infection with hepatitis B and C viruses (HBV/HCV) in HCC has been well documented all over the world and, taken together, these viral infections are present in more than 80% of new primary liver cancer cases (Tsukuma et al., 1993, Brechot et al., 1998). In addition, at least in some geographical areas, chemical carcinogenic compounds (essentially alcohol and aflatoxin B1) represent major risk factors of primary liver cancer (Grisham, 1996). The available data in Europe suggests a further important rise in HCC incidence. This, as well as the major role of hepatitis viruses in this cancer, put forward this tumor as a major health care problem.

Until recently, the genetic abnormalities occuring in HCC were poorly defined. Indeed like a majority of epithelial tumors, HCC is highly refractory to classical cytogenetic analysis. In addition, the lack of specific translocations and the complexity of the genomic rearrangements found in each solid tumor have been responsible for the slow progress in the genetic analysis of this particular type of neoplasia. Consequently, only a handful of tumor suppressor genes (TSG) or protooncogenes potentially implicated in liver carcinogenesis have been carefully studied: CDKN2A (in 9p21 on human genome), H/K/N-RAS (11p15, 12p12, 1p13, respectively), PTEN (10q23) (Challen et al., 1992, Biden et al., 1997, Yao et al., 1999), p53 (17p13), and the b-catenin gene (3p21). The latter two are consistently found mutated in 30% (Bressac etal., 1991) and 20% (de la Coste etal., 1998, Miyoshi et al., 1998, Terris et al., 1999) of HCC, respectively. A putative tumor-suppressive function in liver of the product of the mannose-6-phosphate/insulin-like growth factor-2 receptor (M6P/IGF2R) gene (located on chromosome 6q25-q26) remains controversial (DeSouza et al., 1995, Wada et al., 1999).

### SUMMARY OF THE INVENTION

Taking advantage of the development of high-density microsatellite maps of the human genome, we decided to perform an allelotype, i.e a genome-wide scanning for loss of heterozygosity (LOH) in HCC. According to the theory put forward by W. K. Cavenee in 1983 for the RB gene and double-checked in many instances afterwards, at least one copy of a TSG is inactivated by allelic loss (Cavenee et al., 1983). Therefore, a LOH assay, which detects allelic deletions affecting tumor DNA, is of great value for locating candidate genes implicated in cancer development (Vogelstein et al., 1989). An aim of this invention is to perform in priority the positional cloning of TSG located on chromosome 8p in a first instance and subsequently in chromosomes 4q and 13q.

### DETAILED DESCRIPTION OF THE INVENTION

The human cell has 23 pairs of chromosomes. Every cell has two copies of a gene. Each copy is located on a chromosome in a chromosome pair, called a gene pair. Alleles are alternative forms of a gene. When a cell has two different alleles that constitute the gene pair, it is considered to be heterozygous for that gene. In contrast, when a cell has identical alleles that constitute a gene pair, it is considered to be homozygous. An allelotype is a global profile of a chromosome(s) that identifies the alleles present for each gene analyzed on the chromosome(s). In this invention, a genome-wide scanning was done. In some cases, a heterozygous person can have one dominant normal allele that masks the effect of a second mutated, cancerous allele in the gene pair. As with the case of Rb (retinoblastoma), loss of the normal allele, and thus loss of heterozygosity, results in a cessation of the masking effect. This allows the cancerous trait to emerge, which makes it possible to look for a loss of heterozygosity in identifying and locating (mapping) potential tumor suppressor genes responsible for hepatocellular carcinoma.

The collection of samples analyzed was mainly composed of HBV/HCV-related HCCs from European and Asian patients. DNA samples from 120 tumors and their non-tumorous counterparts were genotyped with a set of 200 microsatellite markers distributed throughout the chromosomes with a mean interval of 20 centimorgans (cM). A genotype is a global assessment of the genes present and their allelic forms in an organism's genome (genetic material). Present on every chromosome are a series of landmarks, or genetic locations (genetic loci) of known genes. When a genetic map is generated, one can localize unknown regions by linking them to positions of the known genetic markers by methods of genetic analysis known to those of ordinary skill in the art. The closer these markers are situated to each other, the more accurate the genetic map will be as the linkage of the genes will be better defined. A centimorgan defines a particular distance on the chromosome.

More particularly, a dozen frequently deleted regions were evidenced, mainly on 8p23, 4q21-23 and 4q35, as well as on 1p, 6q, 13q, 14q, 16pq, and 17p. While some common LOH regions colocalize with already identified tumor suppressors, like p53 on 17p, it is remarkable that the liver TSG present in the most prominent regions 8p, 4q, 13q, and 6q still remain to be identified (Nagai et al., 1997). A comparable study performed by G. Thomas and collaborators showed similar results (Boige et al., 1997).

In parallel, we used comparative genomic hybridization (CGH), a newly described molecular cytogenetic technique, to detect and map genomic aberrations in 50 HCCs from patients chronically infected with HBV. Comparative genomic hybridization produces a map of DNA sequence copy number as a function of chromosomal location throughout the entire genome. Differentially labeled test DNA and normal reference DNA can be hybridized simultaneously to normal chromosome spreads. The hybridization can be detected with two different fluorochromes or markers that fluoresce in different colors. Regions of gain or loss of DNA sequences, such as deletions, duplications, or amplifications, can be seen as changes in the ratio of the intensities of the two fluorochromes along the target chromosomes. Unlike the CGH, which assesses type of genetic alteration (i.e. a loss or gain) material has occurred at a locus (gene location), the LOH reports genotype (heterozygous v. homozygous), but not any additional information on specific changes to the chromosomal structure. CGH clearly differentiates, among the copy number alterations, the amplifications from the losses. Accordingly, this method allows a qualitative identification of chromosome copy number changes in opposition to the usual LOH assay, which detects allelic imbalances (Kallioniemi et al., 1992). In particular, using CGH, observed losses were most prevalent on chromosome regions 4q, 8p, 16q, 17p, 13q, 6q, and 1 p. Frequent gains occured on 8q, 1 q, 6p, and 17q suggesting the presence, in these regions, of genes conferring a growth advantage to liver tumor cells. This study, which confirmed and extended the LOH data, made it possible to definitely discriminate between deletions and amplifications (Marchio et al., 1997).

The highest rate of LOH detected in the original allelotype involved a microsatellite marker located on chromosome 8p23 (D8S277, 42% of informative cases). Loss of the 8p region is common to many tumor types and this alteration is as frequent as 1 p or 17p deletions in epithelial tumors. Deletions of 8p markers have been frequently observed in breast, colorectal, ovary, and prostate cancer or head and neck squamous cell carcinoma (Pineau et al., 1999 and references therein). Consequently, a secondary allelotyping was conducted on chromosomal arm 8p to better define the regions of preferential loss, which might carry putative tumor suppressor genes. Some of the genetic markers (described above) demonstrate a large variability in the number of different alleles that can constitute that marker. Such variance in a gene population is called a polymorphism. This analysis looks at several of these markers that are a very short distance from one another (2 cM), yielding a high-density readout of the markers in that region of chromosome 8p.

A high-density polymorphic marker analysis of 109 paired normal and primary liver tumor samples was then undertaken using 40 microsatellites positioned every 2 cM in average throughout 8p. The data confirmed that 8p loss is a major genetic event, occuring in 60% of the tumors at one or more microsatellite loci. Three distinct minimal deleted areas were defined: a 13 cM region in the distal part of 8p21, a 9 cM area in the more proximal portion of 8p22 and a 5 cM area in 8p23. These data strongly suggest the presence on 8p of at least one, and maybe three, novel TSG influential in HCC development (Pineau et al., 1999).

An alternative approach to the fine deletion mapping using the LOH assay consists in a search for homozygous deletions (HD) on 8p. A deletion is the removal or the absence of a genetic sequence from the chromosome. Homozygous deletions occur when both genes in a gene pair have been deleted. A collection of 110 tumor cell lines was obtained. More than half of them were from hepatobiliary origin. The remaining were established from some of the tumor types exhibiting high rates of LOH on chromosome 8p, i.e. breast, ovarian, head, and neck squamous cell carcinomas, as well as non-small- and small-cell lung cancers. In contrast to tumorous samples obtained ex vivo, tumor cell lines are not contaminated with normal cells and represent consequently a biological material of choice for HD detection. ESTs, STRPs and STSs represent different types of unique genetic targets that lie throughout the chromosome. These targets can be assessed for the complete absence of the target, a homozygous deletion, or an actual sequencing of the target to determine mutations that have occurred. The complete loss of the sequence has the same global effect as a loss of heterozygosity in that the function of the normal wild type gene is lost. This invention concentrates on targets that lie in loci (locations) of chromosome 8p identified as experiencing deletions. Expressed Sequence Tags are small areas of known DNA sequence that lie in genes that are actively expressed in the cell. Simple Tandem Repeat Polymorphisms are small areas in the chromosome in which a sequence has been duplicated, wherein the repeats lie next to each other. Short Tagged Sequences represent known areas of unique human DNA sequence. Cell lines were screened by STS marker scanning, i.e. for the presence of amplification products (amplimers) for Expressed Sequence Tags (ESTs), Simple Tandem Repeat Polymorphism (STRP), and Short Tagged Sequences (STS) located every 250 kb in the critical loci of deletions (Pineau et al., 1999). An absence of amplimer in such regions, hallmark of a HD, would be indicative of the presence of a candidate TSG.

The search for HD using standard PCR or substractive techniques as Representational Difference Analysis (RDA) led in the recent past to the identification of a number of TSG implicated in other cancers (Lisitsyn et al., 1993). Among them, one can cite CDKN2A, BRCA2, FHIT, DPC4, PTEN, and hSNF5. These bona fide tumor suppressor genes are involved in the development of various solid tumors, such as melanomas, breast, gastrointestinal, brain, and paediatric rhabdoid tumors, respectively (Kamb et al., 1994, Wooster et al., 1995, Hahn et al., 1996, Ohta et al., 1996, Li et al., 1997, Versteege et al., 1998). Representational Difference Analysis (RDA) is a method in which one can amplify out sequences that are deleted in an abnormal (i.e. cancerous) genome. For example, to ask if there are sequences deleted from chromosome 8p HCC cell line, one would isolate the DNA from that cell line and isolate the DNA from a normal cell line of the same lineage (i.e. liver cells). DNA sequences that are in common between the two cell lines will hybridize. DNA sequences present in the normal DNA but are deleted in the abnormal DNA will have nothing to hybridize to. The hybridized sequences are subtracted out, leaving only the unbound sequences that are deleted in the abnormal DNA that are subsequently amplified for further identification.

The search for HD in HCC on 8p as well as on other chromosomes has not been seriously undertaken so far. However, a few HD have been already found on chromosome 8p in 3 tumor types: prostate, pancreas, and squamous cell carcinoma (CC). Accordingly, different types of biological specimens were used, and different segments of chromosome 8p were found homozygously deleted (cf Table 1 below).

TABLE 1

| | | Homozygous | | | |
|---|---|---|---|---|---|
| | | Deletion | | | |
| Tissues | Samples | cytogenetics | loci involved | app. size | Author |
| Prostate | tumors | 8p22 | D8S1991-1992 | 750 kb | (Bova et |
| | | | | | al., 1996) |
| Prostate | tumors | 8p12 | D8S87 | <1400 kb | (Prasad et |
| | | | | | al., 1998) |
| Prostate | cell-lines | 8p12-21 | D8S1769-2162 | 730-1320 | (Van |
| | | | | kb | Alewijk et |
| | | | | | al., 1999) |
| Pancreas | cell-lines | 8p22 | D8S1991-1992 | 450 kb | (Levy et al., |
| | | | | | 1999) |
| Squamous CC | tumor | 8p23 | D8S262-1806 | ? | (Ishwad et |
| | | | | | al., 1999) |
| Squamous CC | cell-lines | 8p23 | D8S1788-1824 | ? | (Sun et al., |
| | | | | | 1999) |

Legend of Table 1: CC, cell carcinoma; kb, kilobases, app size, approximate size.

The 8p STS mapping in the minimal regions of loss in 8p21, 8p22 and 8p23 (10, 8 and 5 megabases, respectively) were selected on a chromosome 8 contig established by the University of Southampton (United-Kingdom, ftp://cedar.genetics.soton.ac.uk/pub/chrom8/). This high-density contig is a quasi-exhaustive compendium of available markers (EST, STRP, and STS) mapping on chromosome 8. Some additional public markers were added to our study (as D8S1991, D8S1992). The average density of STS, on this contig is 200 kb. Such a tool associated to our large collection of tumor cell lines was appropriate to detect small HD in our 3 regions of interest.

The locations of these STS targets were identified (mapped) in three specific areas of chromosome 8 identified as having deletions associated with HCC. Chromosome nomenclature follows a specific pattern. The first number identifies the chromosome (8), the first letter identifies the arm of the chromosome (p) which could be p or q, and the next number identifies the specific position on the arm (i.e. 21, 22 or 23). The physical size of each area on the chromosome is given in megabases. Each megabase is 1,000,000 bases or nucleotides in the DNA. For example, 8p21 is 10⁷ bases long. A contig is a contiguous map of these STS targets in chromosome 8, giving their location on the chromosome and distance from each other. The average space between each target or marker is only 200 kilobases or 2×10⁵ bases, which is closely spaced and thus high-density. The closer these markers are to each other, the more accurately one can map the location of a deletion or sequence change on the chromosome. In addition, the smaller the sequence between markers, the easier it is to detect small deletions between those markers. Thus, the more markers that are known, the better.

False positive results (i.e absence of amplimeres) attributable to polymorphisms in primer sequence will be ruled out by the design of new primers on the same locus. Subsequently to HD detection, their sizes and borders will be determined by fluorescent in situ hybridization (FISH) on interphase nuclei using probes prepared from YAC, BAC or PAC containing the deleted amplimers. This experiment will allow us to further reduce the size of the chromosome segment to study.

Tumor suppressor genes often function as regulators of normal cellular processes. Mutations can occur in one copy of a gene in one chromosome while the other copy in the sister chromosome (of the chromosome pair) remains normal. This heterozygous state, if the cancerous trait is recessive, will result in normal function because at least one copy of the normal gene is present. If however, via cell division or some other process, the normal copy is lost (a loss of heterozygosity), then only the mutated and nonfunctional copy remains and is expressed. Another genetic mechanism that attains the same result is homozygous deletion wherein both copies of the gene are deleted from the chromosome, a homozygous deletion. The end result is identical to a loss of heterozygosity in that the function of the normal gene is lost but in this case it is not replaced with anything. This loss of the normal protein results in a loss of the regulator function of the TSG, allowing normal cellular processes (such as cell division) to proceed unchecked. The result is cancer.

This invention identifies regions of chromosomes that sustain a loss of heterozygosity or a homozygous deletion and that are associated with hepatocellular carcinoma. Initially techniques such as allelotyping and comparative genomic hybridization were used on HCC tumor samples to broadly identify the locations of LOH and HD down to the specific chromosome and to which arm of the chromosome the alteration maps. Among the areas identified using these techniques was chromosome 8, arm p (8p). Because of the high frequency of LOH and its association with a variety of other cancers and tumors, the focus was on 8p to produce a finer, more detailed map of the deletions present. To this end, high-density polymorphic marker analysis and STS marker scanning were used to identify deletions in three regions of 8p, those being 8p21, 8p22, and 8p23. In the process, a very useful tool for future analysis of 8p is provided. A fine genetic map of several specific markers located in 8p that are closely spaced together have been identified..

This body of work represents the identification of one or more TSG involved in the development of HCC. As the invention stands, these specific deletions, to the detail presented here, can be used as genetic markers to diagnose the propensity of a patient for developing HCC. The addition made by the invention to the contig of 8p results in a very detailed map of several marker types. Such a map is very useful to others interested in doing different types of cytogenetic analysis on this region of chromosome 8.

Materials and Methods

Human Cell Lines and DNA Extraction

A total of 95 human cell lines included 58 hepatobiliary and 37 non hepatobiliary cell lines were extensively cultured to 30 million of cells. High molecular weight genomic DNAs were extracted and purified as described previously by sambrook et al. (Sambrook et al., 1989). DNAs were quantified in an optic densitometer and homogeneously diluted to a concentration of 50 ng/ml.

PCR Analysis

The 95 human cell lines were assayed for deletion by PCR with 43 primer pairs located in the MRL3 region between D8262 and D8S1825. The primers have been choosen to be distributed every 250 kilobases in this region. The sequences of the oligonucleotides and their distribution are freely publicly available on the electronic databases, particularly on the Internet World Wide Web at the following address:“http://cedar.genetics.soton.ac.uk/pub/chrom8/map.html”. It contains microsatellite as well as STSs markers (Simple Tag Sequences) distributed as follows:

D8S262

D8S1824

D8S201

D8S7

D8S1042

D8S1140

D8S1806

D8S1099

D8S307

D8S1798

D8S518

D8S1963

D8S1788

D8S1781

WI-22787 (renamed STS302)

248WA9 (renamed STS315)

D8S1742

N54165 (renamed STS304)

A008R37 (renamed STS303

STSG43139 (renamed STS305)

AA011655 (renamed STS306)

D8S561

STSG30148 (renamed STS307)

STSG12852(renamed STS308)

STSG39654 (renamed STS309)

STSG2176 (renamed STS310)

A005M25 (renamed STS314)

STSG16046 (renamed STS311)

STSG42716 (renamed STS312)

D8S277

STSG10291 (renamed STS313)

STSG3758 (also D8S141)

D8S1511

D8S1819

D8S1706

D8S1935

D8S439

D8S252

D8S516

D8S503

D8S1469

D8S1825

D8S349

PCRs were performed in a final 25 ml reaction volume including 50 ng of genomic DNA, 20 pmol of each primer, 1.25 mM dNTPs, 1 unit of Taq polymerase and 1× PCR buffer (10 mM Tris (pH 8.9), 0.1% Tween 20,1.5 mM MgCl2, 50 mM KCl). An initial denaturation step was performed during 3.5 min at 94° C. Amplification were carried out during 35 cycles of denaturation (94° C. for 45 sec) and annealing (48 to 58° C.. for 1 min) and an elongation (72° C. for 1 min). At the end of the last cycle, samples were incubated for 4 min for complete elongation. PCRs reaction were loaded on an 2% agarose gel containing 0.5 mg/ml of ethidium bromide.

The presence or absence of an amplified products were estimated on an UV band luminometer. Each negative sample was reamplified in the same condition. If the second amplification confirmed the absence of PCR product, nested primers of the corresponding fragment are designed and tested on the negative DNAs.

| | | |
|---|---|---|
| D8S503bp: | 5′GTTCAAATTGTCTCTAATGG 3′ | [SEQ ID NO: 1 ] |
| D8S503bj: | 5′CTTACACATCGCTCAGAAAC 3′ | [SEQ ID NO: 2 ] |
| D8S262bs: | 5′CTTGTATGTATATAAACGCC 3′ | [SEQ ID NO: 3 ] |
| D8S262bj: | 5′GCTGATCATGGTACCACATG 3′ | [SEQ ID NO: 4 ] |
| D8S1824bp: | 5′CTTCCAGCGTTTATTGCATC 3′ | [SEQ ID NO: 5 ] |
| D8S1824bj: | 5′TTGCCAGTCAGTATGTCAAG 3′ | [SEQ ID NO: 6 ] |
| D8S1788bp: | 5′CATTAAATTTGTAGCTACAG 3′ | [SEQ ID NO: 7 ] |
| D8S1788bj: | 5′TTTTCACTATGCGTGCATAC 3′ | [SEQ ID NO: 8 ] |
| STS303be: | 5′GATCTAGATGAAGAAATGG 3′ | [SEQ ID NO: 9 ] |
| STS303bj: | 5′CAAATACTTAGAATCATCC 3′ | [SEQ ID NO: 10 ] |
| D8S1781Bp: | 5′ACAGGGGTGACACTTCACAG 3′ | [SEQ ID NO: 11 ] |
| D8S1781Bj: | 5′ATGTTCACATCTCCTGAAGC 3′ | [SEQ ID NO: 12 ] |
| STS303CE: | 5′AAGAAGTGCAGAAGGAAG 3′ | [SEQ ID NO: 13 ] |
| STS303dj: | 5′CTAGATGAAGAAATGGGG 3′ | [SEQ ID NO: 14 ] |

Only the Li7A hepatobiliary cell line presented a deleted region included the D8S262, D8S1824, D8S1781, D8S1788 markers. These four markers, as well as D8S201, D8S1798, D8S 1806 and D8S264, were tested on 22 additional non hepatobiliary cell lines. No deletion have been observed.

In reference to the 8p map disposible on “http://cedar.genetics.soton.ac.uk/pub/chrom8/map.html” World Web Site, the D8S262, D8S1824, D8S1781, D8S1788 markers did not appear to be connected. The sequences of the four amplified products have been compared to the htgs unfinished High Throughput Genomic Sequences using the “http://www.ncbi.nim.nih.gov/blast/blast.cgi” Internet World Wide Web site. Only the D8S262 marker showed a 100% homology with the sequence of an human BAC clone named 188e04 located on 8p23, sequenced by the genome Sequencing Centre at the Institute of Molecular Biotechnology, lena, Germany. The freely publicly available complete sequence of the BAC clone 188e04 allowed us to designe primers in an unique region located near the ends of the BAC to confirmed that the proximal region of D8S262 is lost and if the other end, distanced of 170028 bp, extends the deleted region in the Li7A cell line. The sequences of the primers are:

| | | |
|---|---|---|
| 188e4-Nf: | 5′GGAGTGAGTCCAGAGATTCT 3′ | [SEQ ID NO: 15 ] |
| 188e4-Nr: | 5′TCACACAGGATTTCAACAGA 3′ | [SEQ ID NO: 16 ] |
| 188e4-Cf: | 5′TCACAGGTAAAGGCTGAAG 3′ | [SEQ ID NO: 17 ] |
| 188e4-Cr: | 5′AGAAGGGCAATCTGTGAGTA 3′ | [SEQ ID NO: 18 ] |

Both ends of the 188e04 BAC clone are lost in the Li7A cell line extending the deleted region of 168511 bp. Comparison of the 188e04 sequence with the Data Bank of the htgs unfinished High Throughput Genomic Sequences on the “http://www.ncbi.nlm.nih.gov/blast/blast.cgi” Web site revealed that the distal region is D8S262 is highly homologous to an other BAC clone named 315_I_{—}17 sequenced by the Whitehead Institute/MIT Center for Genome Research, USA. The freely publicly ‘working draft’ sequence of this BAC is constituted of 9 contigs.

| | | |
|---|---|---|
| 1 | 9370: | contig of 9370 bp in length |
| 9371 | 12536: | contig of 3166 bp in length |
| 12537 | 22556: | contig of 10020 bp in length |
| 22557 | 33359: | contig of 10803 bp in length |
| 33360 | 49987: | contig of 16628 bp in length |
| 49988 | 71597: | contig of 21610 bp in length |
| 71598 | 102069: | contig of 30472 bp in length |
| 102070 | 169862: | contig of 67793 bp in length |
| 169863 | 174219: | contig of 4357 bp in length. |

The true order of the contigs is not known and their order in this sequence record is arbitrary. the exact sizes of the gaps between the contigs are unknown.

The contig: 49988; 71597 of 21610 bp in length corresponded to the last fragment containing 188e04 sequences . A primer pair located at the ends of the 315_I17 BAC clone were designed:

| | | |
|---|---|---|
| 315I17aS | gagggcactttctttgtatg | [SEQ ID NO: 19 ] |
| 315I17aAS | cccaaggtatatttcctcct | [SEQ ID NO: 20 ] |

The corresponding amplified sequence is not present in the Li7A cell line, extending the deleted region of 67788 bp.

In parallel, a BAC clone named 2003M15, has been freely provided by the genome Sequencing Centre at the Institute of Molecular Biotechnology, Iena, Germany, as overlaping the 188e04 clone in the orientation than the 315-117 clone.

A recent publication by Sun and al. (Sun et al., 1999) ( disposible at Pasteur Institut library on the of Jan. 28, 2000) concerning the identical region, determined a panel of new STS markers defined by their BAC contig. These markers: 341 B24-T7, 341 B24-SP6, 309K3-T7, 309K3-SP6, 370L3-T7, 370L3-SP6, 56309-T7, 459J20-SP6, 389E23-T7, 389E23-SP6, 254M4-T7, 254M4-SP6, 236F7-T7, 549J13-T7 have been tested on the Li7A cell line. All the markers located upstream D8S1824 are retained in Li7A restricted the telomeric boundary closed to D8S1824.

In the centromeric region, all the markers located downstream D8S262 and contained in the 188e04 BAC clone are lost, confirming previous data obtained by PCR analysis. The centromeric boundary of the deleted region in Li7A has been narrowed down using new primers generated from the sequences of the two contigs 33360; 49987 of 16628 bp in length and the 102070; 169862: of 67793 bp in length from the 315_I17 BAC clone, contigs that did not overlap with the 188e04 BAC clone. The generated primer pair sequences are:

| | | |
|---|---|---|
| 315I17fg5f | ATTGCAGAGAGTAAGGCAAA | [SEQ ID NO: 21 ] |
| 315I17fg5r | GCTGAAAGCTCTAAACAGGA | [SEQ ID NO: 22 ] |
| 315I17fg8Nf | CGGTTTGCCAGTATTTTAT | [SEQ ID NO: 23 ] |
| 315I17fg8Nr | TACCAGAGGTACAAGGAGGA | [SEQ ID NO: 24 ] |
| 315I17fg8Cf | TGTAGTCCAGGTCTGAGCTT | [SEQ ID NO: 25 ] |
| 315I17fg8Cr | TAAACGTAGTCCTTGTGGCT | [SEQ ID NO: 26 ] |
| 315I17fg8Af | AAGGAAAAAGTGAGAGGACC | [SEQ ID NO: 27 ] |
| 315I17fg8Ar | CCTGTAGTCCCAGCTACTTG | [SEQ ID NO: 28 ] |
| 315I17fg8Bf | AACTATTAACGGCTGTTTGC | [SEQ ID NO: 29 ] |
| 315I17fg8Br | TCTATTTCTGGGGCATCTTA | [SEQ ID NO: 30 ] |
| 315I17fg8Df | ATACAGCAATGTGCTCTCCT | [SEQ ID NO: 31 ] |
| 315I17fg8Dr | AGTGGGGGTAGGAGTAATGT | [SEQ ID NO: 32 ] |
| 315I17fg8Ef | CTGTGGTTGGAAGTCAGATT | [SEQ ID NO: 33 ] |
| 315I17fg8Er | GAGCTGGTTACACAAAGAGG | [SEQ ID NO: 34 ] |
| 315I17fg8Ff | ACATTCTCTCCAAACAGTGG | [SEQ ID NO: 35 ] |
| 315I17fg8Fr | CACCCTACCTCATCCAATTA | [SEQ ID NO: 36 ] |

The amplimer obtained with the sequence using the 315117fg8B sets corresponds to the most proximal locus involved in the homozygous deletion in Li7A cell line. The total size of the homozygously deleted region in Li7A flanked by the 370L3SP6 and 315117fg8D loci markers represents a maximum of 345 Kb. Relevant references are Sambrook, J., Fritsch, E. and Maniatis, T. (1989) Molecular cloning: A Laboratory Manual. 1st Ed. Cold Spring Harbor, N.Y./Cold Spring Harbor Laboratory Press; and Sun, P. C., Schmidt, A. P., Pashia, M. E., Sunwoo, J. B. and Scholnick, S. B. (1999) Homozygous deletions define a region of 8p23.2 containing a putative tumor supressor gene. Genomics, 62, 184-188.

The inventors have identified the location of deletions containing the tumor suppressor gene in region 8p23. Several probes (amplifiers) that span the deletions containing the tumor suppressor gene, primers for amplifying the deletion containing the tumor suppressor gene and primers for amplifying r eg ions of the chromosome that abut the deletion have also been designed.

Nuclear acid probe, 544 pb
:Xd71CX12
ATgACAGATACGATTTAATCGAcTCCACTaTAaGGATTGCCTcGAAGGCAAGa
ATTCGGGCaCGAGGGgATTCTTAGAgA
TCAGgTGGcaCGAAAGCTCcATCATAtGGcTAAcTGGGCATAaCCCTcccccCCCT
CAGTATCAGAGCAAGaATTGGCTa
CGAcTTCCATTCACTctTGCAGCAACACCCGACGCaAAGATTAACGCTcCAGTC
CAAGTGAAAAAGGCGATTGAGTTGAA
GTCAAGAGGAGTCAAGATGCTGCCCAGCAAGGATGGAAGCCATAAAAACTC
TGTCTGGCATCAGCAAGAGTTCAGCAAGT
GCAGGAAGAAAAAGAGAGAGATCATGACAAGGAATGGGAGAATTTCCCTG
ACAGCCTCAGGAAACTTGCAGTTTGATAAT
TAAACAGATCAAGGTCACTCAGATGAGCTGATGGGACATGCTGTGTACGGA
GGAGCATTTGCAGTTACAACACTTTGTAG
CCATGCAGGATGGGGCAATTAATCCAGAACCATTATTTaataaaAAGATGATTT
TTTAAATGTG
Primers allowing of the detection of deletions (double deletions)

| | |
|---|---|
| D8S262BS: | 5′CTTGTATGTATATAAACGCC 3′ |
| D8S262BJ: | 5′GCTGATCATGGTACCACATG 3′ |
| D8S1824BP: | 5′CTTCCAGCGTTTATTGCATC 3′ |
| D8S1824BJ: | 5′TTGCCAGTCAGTATGTCAAG 3′ |
| D8S1788BP: | 5′CATTAAATTTGTAGCTACAG 3′ |
| D8SI788BJ: | 5′TTTTCACTATGCGTGCATAC T |
| D8S1781BP: | 5′ACAGGGGTGACACTTCACAG 3′ |
| D8S1781BJ: | 5′ATGTTCACATCTCCTGAAGC 3′ |
| 188e4-Nf: | 5′GGAGTGAGTCCAGAGATTCT 3′ |
| 188e4-Nr: | 5′TCACACAGGATTTCAACAGA 3′ |
| 188e4-Cf: | 5′TCACAGGTAAAGGCTGAAG 3′ |
| 188e4-Cr: | 5′AGAAGGGCAATCTGTGAGTA 3′ |

Amplimers with the breakpoint proximal (have 500 Pb near) double deletions
(primers that abut the deletion):

| | |
|---|---|
| 315I17fg8Df | ATACAGCAATGTGCTCTCCT |
| 315117fg8Dr | AGTGGGGGTAGGAGTAATGT |

Amplimers with the breakpoint distal: (sequence not yet published)

| | |
|---|---|
| 370L3Sp6f | GAGGATGTGACAGTATTGGATATCA |
| 370L3Sp6r | GCCTTCCTATGAGCTGCACGG |

### REFERENCES

The following publications have been cited herein, and the entire disclosure of each publication is relied upon and incorporated by reference herein.

1. Biden, K., Young, J., Buftenshaw, R., Searle, J., Cooksley, G., Xu, D. -B. and Leggett, B. (1997) Frequency of mutation and deletion of the tumor suppressor gene CDKN2A (MTS1/p16) in hepatocellular carcinoma from an Australian population.Hepatology, 25, 593-597.

2. Boige, V., Laurent-Puig, P., Fouchet, P., Fléjou, F., Monges, G., Bedossa, P., Bioulac-Sage, P., Capron, F., Schmitz, A., Olschwang, S. and Thomas, G. (1997) Concerted non syntenic allelic losses in hyperploïd hepatocellular carcinoma as determined by a high-resolution allelotype.Cancer Res., 57,1986-1990.

3. Bova, G., MacGrogan, D., Levy, A., Pin, S., Bookstein, R. and Isaacs, W. (1996) Physical mapping of chromosome 8p22 markers and their homozygous deletion in a metastatic prostate cancer.Genomics, 35, 46-54.

4. Bréchot, C., Jaffredo, F., Lagorce, D., Gerken, G., zum Büschenfelde, K., Papakonstontinou, A., Hadziyannis, S., Romeo, R., Colombo, M., Rodes, J., Bruix, J., Williams, R. and Naoumov, N. (1998) Impact of HBV, HCV and GBV-C/HGV on hepatocellular carcinomas in Europe: results of a European concerted action.J. Hepatol, 29,173-183.

5. Bressac, B., Kew, M., Wands, J. and Ozturk, M. (1991) Selective G to T mutations of p53 gene in hepatocellular carcinoma from southern Africa.Nature, 350, 429-431.

6. Cavenee, W. K., Drya, T. P., Phillips, R. A., Benedict, W. F., Godbout, R., Gallie, B. L., Murphee, A. L., Strong, L. C. and White, R. L. (1983) Expression of recessive alleles by chromosomal mechanisms in retinoblastoma.Nature, 305, 779-784.

7. Challen, C., Guo, K., Collier, J., Cavanagh, D. and Bassendine, M. (1992) Infrequent point mutations in codons 12 and 61 of ras oncogenes in human hepatocellular carcinomas.J Hepatol, 14, 342-346.

8. de la Coste, A., Romagnolo, B., Billuart, P., Renard, C. A., Buendia, M., Soubrane, O., Fabre, M., Chelly, J., Beldjord, C., Kahn, A. and Perret, C. (1998) Somatic mutations of the b-catenin gene are frequent in mouse and hepatocellular carcinoma.Proc. Natl. Acad. Sci. USA, 95, 8847-8851.

9. DeSouza, A., Hankins, G. R., Washington, M. K., Orton, T. C. and Jirtle, R. L. (1995) M6P/IGF2R gene is mutated in human hepatocellular carcinoma with loss of heterozygosity.Nature Genetics, 11, 447-449.

10. Deuffic, S., Poynard, T., Buffat, L. and Valleron, A. (1998) Trends in primary liver cancer.Lancet, 351, 214-215.

11. Dillon, D., Howe, C., Bosari, S. and Costa, J. (1998) The molecular biology of breast cancer: accelerating clinical applications.Crit Rev. Oncogenesis, 9, 125-140.

12. El-Serag, H. and Mason, A. (1999) Rising incidence of hepatocellular carcinoma in the United States.N Engi J Med, 340, 740-750.

13. Grisham, J. W. (1996) Interspecies comparison of liver carcinogenesis.Carcinogenesis, 18, 59-81.

14. Hahn, S., Schutte, M, Hoque, A T, Moskaluk, C A, da Costa, L T, Rozenblum, E, Weinstein, C L, Fischer, A, Yeo, C J, Hruban R H and Kern, S. (1996) DPC4, a candidate tumor suppressor gene at human chromosome 18q21.1.Science, 271, 350-353.

15. Ishwad, C., Shuster, M., Bockmuhl, U., Thakker, N., Shah, P., Toomes, C., Dixon, M., Ferrell, R. and Gollin, S. (1999) Frequent allelic loss and homozygous deletion in chromosome band 8p23 in oral cancer. Int J Cancer, 80, 25-31.

16. Kallioniemi, A., Kallioniemi, O. P., Sudar, D., Rutovitz, D., Gray, J. W., Waldman, J. W. and Pinkel, D. (1992) Comparative genomic hybridization for molecular cytogenetic analysis of solid tumors.Science, 258, 818-821.

17. Kamb, A., Gruis, N., Weaver-Feldhaus, J., Liu, Q., Harshman, K., Tavtigian, S., Stockert, E., Day III, R., Johnson, B. and Skolnick, M. (1994) A cell cycle regulator potentially involved in genesis of many tumor types.Science, 264, 436-440.

18. Kinzler, K. and Vogelstein, B. (1997) Cancer-susceptibility genes: Gatekeepers and caretakers.Nature, 386, 761-763.

19. Levy, A., Dang, U. and Bookstein, R. (1999) High-density screen of human tumor cell lines for homozygous deletions of loci on chromosome arm 8p.Genes Chromosomes Cancer, 24, 42-47.

20. Li, J., Yen, C., Liaw, D., Podsypanina, K., Bose, S., Wang, S. I., Puc, J., Miliaresis, C., Rodgers, L., McCombie, R., Bigner, S., Giovanella, B. C., Ittman, M., Tycko, B., Hishboosh, H., Wigler, M. H. and Parsons, R. (1997) PTEN, a putative protein tyrosine phosphatase gene mutated in human brain, breast and protate cancer.Science, 275,1943-1947.

21. Lisitsyn, N., Lisitsyn, N. and Wigler, M. (1993) Cloning the differences between two complex genomes.Science, 259, 946-951.

22. Makimoto, K. and Higuchi, S. (1999) Alcohol consumption as a major risk factor for the rise in liver cancer mortality rates in Japanese men.Int J Epidemiol, 28, 30-34.

23. Marchio, A., Mehdeb, M., Pineau, P., Danglot, G., Tiollais, P., Bernheim, A. and Dejean, A. (1997) Recurrent chromosomal abnormalities in hepatocellular carcinoma detected by comparative genomic hybridization.Genes Chrom. Cancer, 18, 59-65.

24. Markovic, S., Gadzijev, E., Stabuc, B., Croce, L., Masutti, F., Surlan, M., Berden, P., Brencic, E., Visnar-Perovic, A., Sasso, F., Ferlan-Marolt, V., Pozzi Mucelli, F., Cesar, R., Sponza, M. and Tiribelli, C. (1998) Treatment options in western hepatocellular carcinoma: a prospective study of 224 patients.J Hepatol, 29, 650-659.

25. Miyoshi, Y., Iwao, K., Nagasawa, Y., Aihara, T., Sasaki, Y., Imaoka, S., Murata, M., Shimano, T. and Nakamura, Y. (1998) Activation of the b-catenin gene in primary hepatocellular carcinomas by somatic alterations involving exon 3.Cancer Res., 58, 2524-2527.

26. Nagai, H., Pineau, P., Tiollais, P., Buendia, M. A. and Dejean, A. (1997) Comprehensive allelotyping of human hepatocellular carcinoma.Oncogene, 14, 2927-2933.

27. Ohta, M., Inoue, H., Cotticelli, M. G., Kastury, K., Baffa, R., Palazzo, J., Siprashvili, Z., Mori, M., McCue, P., Druck, T., Croce, C. M. and Huebner, K. (1996) The FHIT gene, spanning the chromosome 3p14.2 fragile site and Renal carcinoma-associated t(3;8) breakpoint, is abnormal in digestive tract cancers.Cell, 84, 587-597.

28. Parkin, D., Pisani, P. and Ferlay, J. (1999) Estimates of the worldwide incidence of 25 major cancers in 1990.Int J Cancer, 15, 827-41.

29. Pineau, P., Nagai, H., Prigent, S., Wei, Y., Gyapay, G., Weissenbach, J., Tiollais, P., Buendia, M. and Dejean, A. (1999) Identification of three distinct regions of allelic deletions on the short arm of chromosome 8 in hepatocellular carcinoma.Oncogene, 18, 3127-3134.

30. Prasad, M., Trybus, T., Wojno, K. and Macoska, J. (1998) Homozygous and frequent deletion of proximal 8p sequences in human prostate cancers: identification of a potential tumor suppressor gene site.Genes Chrom Cancer, 23, 255-262.

31. Sun, P., Schmidt, A., Pashia, M., Sunwoo, J. and Scholnick, S. (1999) Homozygous deletions define a region of 8p23.2 containing a putative tumor suppressor gene.Genomics, 62, 184-188.

32. Taylor-Robinson, S., Foster, G. R., Arora, S., Hargreaves, S. and Thomas, H. C. (1997) Increase in primary liver cancer in the UK, 1979-1994.Lancet, 350, 1142-1143.

33. Terris, B., Pineau, P., Bregeaud, L., Valla, D., Belghiti, J., Degott, C. and Dejean, A. (1999) Close correlation between b-catenin gene alterations and nuclear accumulation of the protein in human hepatocellular carcinoma.Oncogene, 18, 6583-6588

34. Tsukuma, H., Hiyama, T., Tnaka, S., Nakao, M., Yabuuchi, T., Kitamura, T., Nakanishi, K., Fujimoto, I., Inoue, A., Yamazaki, H. and Kawashima, T. (1993) Risk factors for hepatocellular carcinoma among patients with chronic liver disease.N. Engl J Med, 328,1797-1801.

35. Van Alewijk, D., Van derWeiden, M., Eussen, B., LD, V. D. A. -T., Ehren-van Eekelen, C., Konig, J., van Steenbrugge, G., Dinjens, W. and Trapman, J. (1999) Identification of a homozygous deletion at 8p12-21 in a human prostate cancer xenograft.Genes Chromosomes Cancer, 24,119-126.

36. Versteege, I., Sevenet, N., Lange, J., Rousseau-Merck, M., Ambros, P., Handgretinger, R., Aurias, A. and Delattre, O. (1998) Truncating mutations of hSNF5/INI1 in aggressive paediatric cancer.Nature, 394, 203-206.

37. Vogelstein, B., Fearon, E. R., Kern, S. E., Hamilton, S. R., Preisenger, A. C., Nakamura, Y. and White, R. (1989) Allelotype of colorectal carcinomas.Science, 244, 207-211.

38. Wada, I., Kanada, H., Nomura, K., Kato, Y., Machinami, R. and Kitagawa, T. (1999) Failure to Detect Genetic Alteration of the Mannose-6-Phosphate/Insulin-Like Growth Factor 2 Receptor (M6P/IGF2R) Gene in Hepatocellular Carcinomas in Japan.Hepatology, 29,1718-1721.

39. Wooster, R., Bignell, G., Lancaster, J., Swift, S., Seal, S., Mangion, J., Collins, N., Gregory, S., Gumbs, C., Micklem, G., Barfoot, R., Hamoudi, R., Patel, S. and al. (1995) Identification of the breast cancer susceptibility gene BRCA2.Nature, 378, 789-792.

40. Yao, Y., Ping, X., Zhang, H., Chen, F., Lee, P., Ahsan, H., Chen, C., Lee, P., Peacocke, M., Santella, R. and Tsou, H. (1999) PTEN/MMAC1 mutations in hepatocellular carcinomas.Oncogene, 18, 3181-85.

41. U.S. Pat. No. 6,037,121 to Garkavtsev et al., DNA Sequence Encoding a Tumor Suppressor Gene.

What it claimed is:

1. A method for detecting human hepatocellular carcinoma (HCC), wherein the method comprises detecting a homozygous deletion in human chromosome 8p23.
